(19) United States
(12) Patent Application Publication
(10) Pub. No.: US 2024/0398473 A1
(43) Pub. Date: Dec. 5, 2024

(54) DISTAL ASSEMBLY FOR CATHETER WITH LUMENS RUNNING ALONG SPINES

(71) Applicant: Biosense Webster (Israel) Ltd., Yokneam (IL)

(72) Inventors: Justin George LICHTER, Irvine, CA (US); Joseph Thomas KEYES, Sierra Madre, CA (US)

(73) Assignee: Biosense Webster (Israel) Ltd., Yokneam (IL)

(21) Appl. No.: 18/799,583

(22) Filed: Aug. 9, 2024

Related U.S. Application Data
(62) Division of application No. 17/319,957, filed on May 13, 2021, now Pat. No. 12,064,170.

(51) Int. Cl. A61B 18/14; A61B 17/00; A61B 18/00

(52) U.S. Cl. CPC A61B 18/1492; A61B 2017/00526; A61B 2018/00267; A61B 2018/00357; A61B 2018/00577; A61B 2018/1467; A61B 2218/002

(57) ABSTRACT

Medical apparatus includes an insertion tube configured for insertion into a body cavity of a patient and a distal assembly, including a plurality of spines having respective proximal ends that are connected distally to the insertion tube. Each spine includes a rib extending along a length of the spine, a flexible polymer sleeve disposed over the rib and defining a lumen running parallel to the rib along the spine, and one or more electrodes disposed on the sleeve and configured to contact tissue within the body cavity.

### CROSS-REFERENCE TO RELATED APPLICATIONS

This application is a divisional of prior filed U.S. patent application Ser. No. 17/319,957 filed on May 13, 2021 (Attorney Docket No. 253757.000206-BIO6517USNP1), which prior application is hereby incorporated by reference as if set forth in full into this application.

### FIELD OF THE INVENTION

The present invention relates generally to invasive medical equipment, and particularly to apparatus for ablating tissue within the body and methods for producing such apparatus.

### BACKGROUND

Cardiac arrythmias are commonly treated by ablation of myocardial tissue in order to block arrhythmogenic electrical pathways. For this purpose, a catheter is inserted through the patient's vascular system into a chamber of the heart, and an electrode or electrodes at the distal end of the catheter are brought into contact with the tissue that is to be ablated. In some cases, high-power radio-frequency (RF) electrical energy is applied to the electrodes in order to ablate the tissue thermally. Alternatively, high-voltage pulses may be applied to the electrodes in order to ablate the tissue by irreversible electroporation (IRE).

Electrical ablation, whether by RF thermal ablation or IRE, generates excess heat, which can cause collateral damage to tissues in and around the ablation site. To reduce tissue temperature and thus mitigate this sort of damage, the area of the electrodes is commonly irrigated during the ablation procedure. In some catheters, the irrigation is applied through small holes in the electrodes themselves, for example as described in U.S. Pat. No. 8,475,450, which is owned by applicant and incorporated by reference herein.

Some ablation procedures use basket catheters, in which multiple electrodes are arrayed along the spines of an expandable basket assembly at the distal end of the catheter. Various schemes have been described for irrigating such basket assemblies during ablation. For example, U.S. Pat. No. 7,955,299 describes a basket catheter with an outer tubing housing an inner fluid delivery tubing having at least one fluid delivery port. A plurality of spines are each connected at a proximal end of the spines to the outer tubing and at a distal end of the spines to the inner fluid delivery tubing. The inner fluid delivery tubing is operable to be moved in a first direction to expand the spines; and in a second direction to collapse the spines. A porous membrane is provided over at least a portion of the inner fluid delivery tubing. A seal is provided at a proximal end of the porous membrane between the porous membrane and the outer tubing and between the porous membrane and the inner fluid delivery tubing, the seal configured for irrigating between the plurality of spines of the basket catheter while preventing fluid ingress into the outer tubing.

### SUMMARY

Embodiments of the present invention that are described hereinbelow provide improved apparatus for ablating tissue with the body, as well as methods for producing such apparatus.

There is therefore provided, in accordance with an embodiment of the invention, medical apparatus, including an insertion tube configured for insertion into a body cavity of a patient and a distal assembly, including a plurality of spines having respective proximal ends that are connected distally to the insertion tube. Each spine includes a rib extending along a length of the spine, a flexible polymer sleeve disposed over the rib and defining a lumen running parallel to the rib along the spine, and one or more electrodes disposed on the sleeve and configured to contact tissue within the body cavity.

In some embodiments, the spines have respective distal ends that are conjoined at a distal end of the distal assembly, and the ribs are configured to bow radially outward when the distal assembly is deployed in the body cavity, whereby the electrodes contact the tissue in the body cavity. In a disclosed embodiment, the ribs are configured to collapse radially inward so that the spines are aligned along an axis of the insertion tube while the apparatus is being inserted into the body cavity. Additionally or alternatively, the insertion tube includes a flexible catheter configured for insertion into a chamber of a heart of the patient, and the electrodes are configured to contact and apply electrical energy to myocardial tissue within the chamber.

In a disclosed embodiment, the rib includes a metal slat. Additionally, or alternatively, the flexible polymer sleeve includes a thermoplastic elastomer.

In one embodiment, the lumen of each of the spines is in fluid communication with an irrigation manifold running through the insertion tube, and irrigation outlets pass through the flexible polymer sleeve to the lumen in a vicinity of the electrodes, whereby an irrigation fluid passing through the irrigation manifold exits the lumen through the irrigation outlets. Additionally or alternatively, each spine includes a wire running through the lumen and connecting electrically to at least one of the electrodes.

There is also provided, in accordance with an embodiment of the invention, a method for producing a medical device. The method includes forming a plurality of spines by, for each spine, placing a mandrel alongside a resilient rib and molding a flexible polymer sleeve over the rib and the mandrel. After molding the sleeve, the mandrel is removed so that the sleeve contains a lumen running parallel to the rib along the spine. One or more electrodes are fixed to the sleeve of each of the spines. Respective proximal ends of the spines are connected together to a distal end of an insertion tube, which is configured for insertion into a body cavity of a patient.

In a disclosed embodiment, the flexible polymer sleeve includes a thermoplastic elastomer tube, and molding the flexible polymer sleeve includes heating the thermoplastic elastomer tube to a temperature sufficient to cause the thermoplastic elastomer tube to shrink to the shape of the rib and the mandrel.

The present invention will be more fully understood from the following detailed description of the embodiments thereof, taken together with the drawings in which:

### BRIEF DESCRIPTION OF THE DRAWINGS

FIG. 1 is a schematic pictorial illustration showing a system for cardiac ablation, in accordance with an embodiment of the invention;

FIGS. 2A and 2B are schematic side views of a catheter basket assembly in collapsed and expanded configurations, respectively, in accordance with an embodiment of the invention;

FIG. 3 is a schematic side view of a spine of a catheter basket assembly, in accordance with an embodiment of the invention;

FIG. 4 is a schematic pictorial view showing details of a distal part of the spine of FIG. 3, in accordance with an embodiment of the invention;

FIG. 5 is a schematic sectional view of a portion of the spine of FIG. 4 taken along a longitudinal cut, in accordance with an embodiment of the invention;

FIG. 6 is a schematic sectional view showing details of a proximal part of the spine of FIG. 3, in accordance with an embodiment of the invention; and

FIG. 7 is a schematic cross-sectional view of the spine of FIG. 4, in accordance with an embodiment of the invention.

### DETAILED DESCRIPTION OF EMBODIMENTS

For efficient, reliable cooling of an ablation site, it is desirable that the irrigation fluid be targeted specifically at the locations of the electrodes. In basket catheters, however, mechanical constraints and strict size limitations make it difficult to deliver the irrigation fluid along the spines to the electrodes. Although it is possible to spray irrigation fluid toward the electrodes, for example from a manifold within the basket assembly, this approach requires a high irrigation flow rate and even then may not achieve adequate cooling of the tissue.

Embodiments of the present invention that are described herein address this problem by providing methods for forming lumens along the spines of the distal assembly of a catheter, as well as assemblies containing such lumens. Spines with lumens can be formed in the disclosed manner efficiently and reliably, without substantially increasing the dimensions of the spines or altering their mechanical properties. Such lumens can be used not only for conveying irrigation fluid to the locations of the electrodes along the spines but also, additionally or alternatively, for other purposes, such as routing electrical wires along the spines. Although the embodiments described below relate specifically to basket catheters, the principles of the present invention may similarly be applied in other sorts of distal assemblies for medical probes, such as multi-arm catheters.

In the disclosed embodiments, a distal assembly of a medical probe, such as a cardiac catheter, comprises multiple spines having respective proximal ends that are connected distally to an insertion tube, which is configured for insertion into a body cavity of a patient. Each spine comprises a rib, such as a metal slat, extending along the length of the spine. A flexible polymer sleeve is disposed over the rib and defines a lumen running parallel to the rib along the spine. To create the lumen, in some embodiments, a mandrel is placed alongside the rib, the sleeve is disposed over the rib and the mandrel, and the mandrel is then removed, leaving the lumen within the sleeve. One or more electrodes are fixed externally over the sleeve so as to contact tissue within the body cavity.

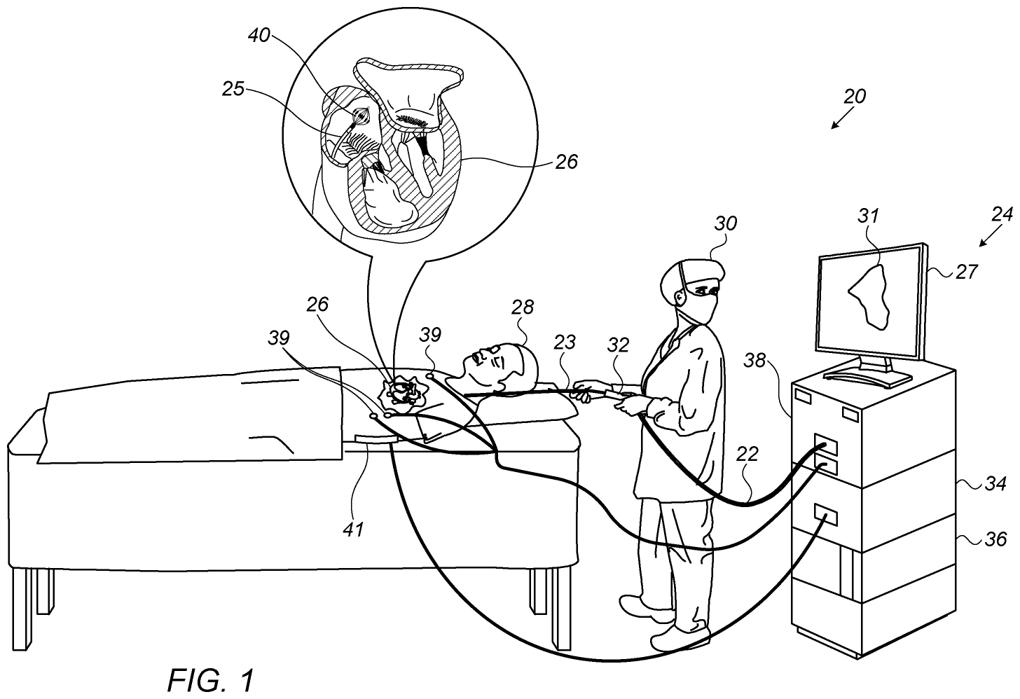

FIG. 1 is a schematic pictorial illustration of a system 20 used in an ablation procedure, in accordance with an embodiment of the invention. Elements of system 20 may be based on components of the CARTO® system, produced by Biosense Webster, Inc. (Irvine, California).

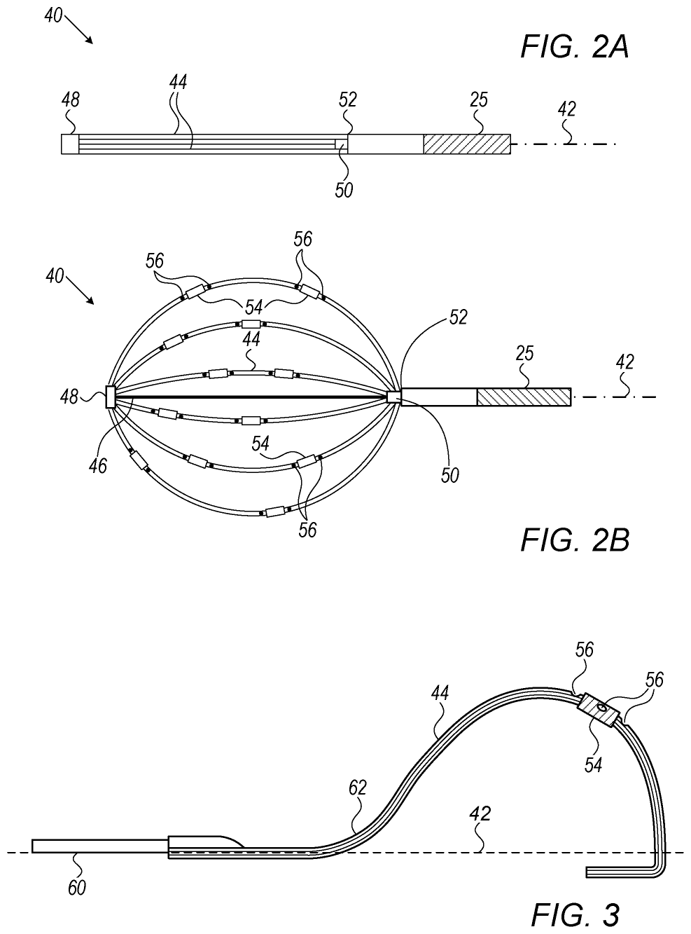

A physician 30 navigates a catheter 22 through the vascular system of a patient 28 into a chamber of a heart 26 of the patient, and then deploys a basket assembly 40 (shown in detail in FIGS. 2A/B) at the distal end of the catheter. The proximal end of basket assembly 40 is connected to the distal end of an insertion tube 25, which physician 30 steers using a manipulator 32 near the proximal end of catheter 22. Basket assembly 40 is inserted in a collapsed configuration through a sheath 23, which passes through the vascular system of patient 28 into the heart chamber where the ablation procedure is to be performed. Once inserted into the heart chamber, basket assembly 40 is deployed from the sheath and allowed to expand within the chamber. Catheter 22 is connected at its proximal end to a control console 24. A display 27 on console 24 may present a map 31 or other image of the heart chamber with an icon showing the location of basket assembly 40 in order to assist physician 30 in positioning the basket assembly at the target location for the ablation procedure.

Once basket assembly 40 is properly deployed and positioned in heart 26, physician 30 actuates an electrical signal generator 38 in console 24 to apply electrical energy (such as IRE pulses or RF waveforms) to the electrodes on the basket assembly, under the control of a processor 36. The electrical energy may be applied in a bipolar mode, between pairs of the electrodes on basket assembly 40, or in a unipolar mode, between the electrodes on basket assembly 40 and a separate common electrode, for example a conductive back patch 41, which is applied to the patient's skin. During the ablation procedure, an irrigation pump 34 delivers an irrigation fluid, such as saline solution, through insertion tube 25 to basket assembly 40.

Typically, catheter 22 comprises one or more position sensors (not shown in the figures), which output position signals that are indicative of the position (location and orientation) of basket assembly 40. For example, basket assembly 40 may incorporates one or more magnetic sensors, which output electrical signals in response to an applied magnetic field. Processor 36 receives and processes the signals in order to find the location and orientation coordinates of basket assembly 40, using techniques that are known in the art and are implemented, for example, in the above-mentioned Carto system. Alternatively or additionally, system 20 may apply other position-sensing technologies in order to find the coordinates of basket assembly 40. For example, processor 36 may sense the impedances between the electrodes on basket assembly 40 and body-surface electrodes 39, which are applied to the chest of patient 28, and may convert the impedances into location coordinates using techniques that are likewise known in the art. In any case, processor 36 uses the coordinates in displaying the location of basket assembly 40 on map 31.

Alternatively, catheter 22 and the ablation techniques that are described herein may be used without the benefit of position sensing. In such embodiments, for example, fluoroscopy and/or other imaging techniques may be used to ascertain the location of basket assembly 40 in heart 26.

The system configuration that is shown in FIG. 1 is presented by way of example for conceptual clarity in understanding the operation of embodiments of the present invention. For the sake of simplicity, FIG. 1 shows only the elements of system 20 that are specifically related to basket assembly 40 and ablation procedures using the basket assembly. The remaining elements of the system will be apparent to those skilled in the art, who will likewise understand that the principles of the present invention may be implemented in other medical therapeutic systems, using other components. All such alternative implementations are considered to be within the scope of the present invention.

FIGS. 2A and 2B are schematic side views of basket assembly 40 in its collapsed and expanded states, respectively, in accordance with an alternative embodiment of the invention. Basket assembly 40 has a distal end 48 and a proximal end 50, which is connected to a distal end 52 of insertion tube 25. The basket assembly comprises multiple spines 44, whose proximal ends are conjoined at proximal end 50, and whose distal ends are conjoined at distal end 48. One or more electrodes 54 are disposed externally on each of spines 44. Irrigation outlets 56 in spines 44 allow irrigation fluid flowing within the spines to exit and irrigate tissue in the vicinity of electrodes 54.

In the collapsed state of FIG. 2A, spines 44 are straight and aligned parallel to a longitudinal axis 42 of insertion tube 25, to facilitate insertion of basket assembly 40 into heart 26. In the expanded state of FIG. 2B, spines 44 bow radially outward, causing electrodes 54 on spines 44 to contact tissue within the heart. In one embodiment, spines 44 are produced such that the stable state of basket assembly 40 is the collapsed state of FIG. 2A: In this case, when basket assembly 40 is pushed out of the sheath, it is expanded by drawing a puller 46, such as a suitable wire, in the proximal direction through insertion tube 25. Releasing puller 46 allows basket assembly 40 to collapse back to its collapsed state. In another embodiment, spines 44 are produced such that the stable state of basket assembly 40 is the expanded state of FIG. 2B: In this case, basket assembly 40 opens out into the expanded stated when it is pushed out of the sheath, and puller 46 may be replaced by a pusher rod to move towards a distal direction and straighten the spines 44 before the sheath is pushed distally to enclose the straightened spines.

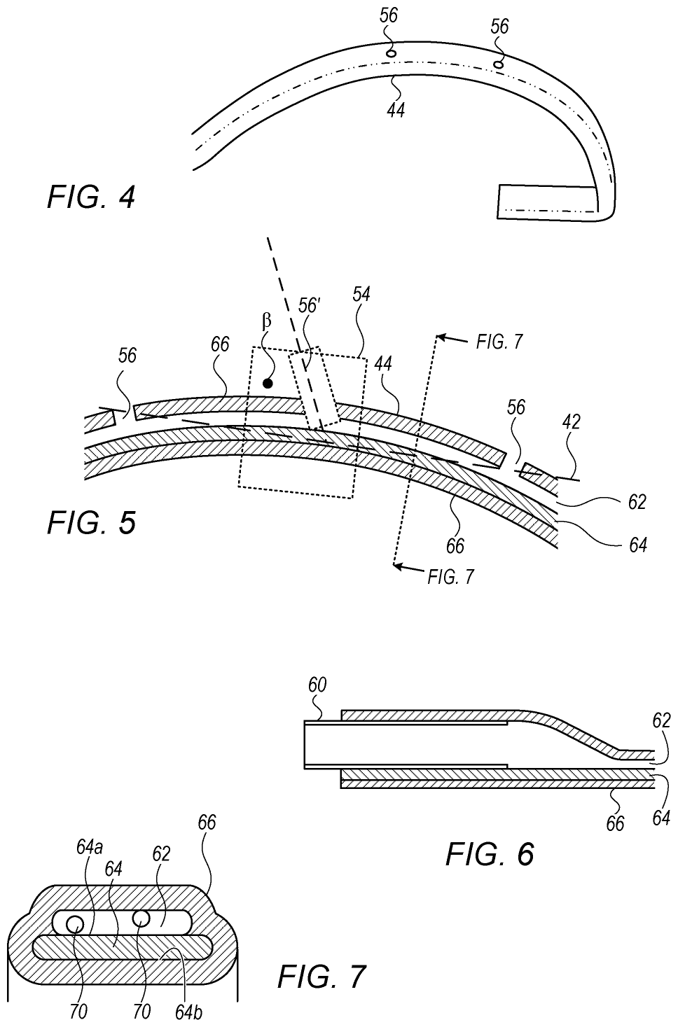

FIGS. 3 and 4 schematically show details of one representative spine 44 in basket assembly 40, in accordance with an embodiment of the invention. FIG. 3 is a side view of spine 44, while FIG. 4 is a pictorial view of spine 44 (without the electrode 44) seen from an angle outside the basket assembly. Electrode 54 is shown in FIG. 3 between irrigation outlets 56 but is omitted from FIG. 4 for visual clarity.

As shown in FIG. 3, a lumen 62 within spine 44 is in fluid communication with an irrigation manifold 60, comprising a tube, which runs through insertion tube 25. Thus, an irrigation fluid that is pumped through irrigation manifold 60 exits lumen 62 through irrigation outlets 56. Alternatively or additionally, as noted earlier, lumen 62 may contain electrical wires 70 (shown in FIG. 7) connecting electrically to electrode 54.

Reference is now made to FIGS. 5-7, which schematically show details of the construction and method of production of spine 44, in accordance with an embodiment of the invention. FIG. 5 is a sectional view of a portion of spine 44 taken along a longitudinal cut (without the electrode 54 for clarity), while FIG. 7 is a cross-sectional view along a radial cut through the spine. FIG. 6 is a sectional view showing details of the proximal part of spine 44, including the connection of lumen 62 to irrigation manifold 60.

Spine 44 comprises a rib 64 running along the length of the spine, with a shape corresponding to the equilibrium shape of the spine. In an example embodiment, rib 64 comprises a relatively rigid slat, such as a long, thin piece of biocompatible material such as, for example, nickel titanium, having the desire curved or straight equilibrium shape. The rib 64 has a first surface 64a and an opposite surface 64b along side (e.g., parallel or non-parallel) with each other. While the embodiment in FIG. 7 shows a rectangular cross section with major side surfaces 64a and 64b, the invention is not limited to such a configuration as long as two major surfaces can be formed that run together (e.g., parallel or non-parallel) to define the slat or rib. Lumen 62 in this embodiment runs along only one of the side surfaces of rib 64, for example along surface 64a as shown in FIG. 7.

To form lumen 62, a mandrel in the shape of the lumen 62 is placed along rib 64, and a thermoplastic elastomer tube or sleeve 66 is fitted over the rib 64 and the mandrel. In one embodiment, the mandrel is made from a flexible, self-lubricating polymer with a high melting temperature, such as polytetrafluoroethylene (PTFE). The elastomer tube or sleeve 66 comprises a biocompatible material with suitable heat-shrinking properties, such as Pebax® polyether block amide shrink tubing. The elastomer sleeve 66 is mounted over rib 64 and the entire assembly is heated to a sufficient temperature to cause the elastomer tube or sleeve 66 to shrink to the shape of the underlying rib 64 and mandrel, thus forming a sleeve 66 with the sort of profile that is shown in FIG. 7. Alternatively, the rib 64 can be placed into a mold and a thermoplastic material can be used in conjunction with the mold to form sleeve 66 as a molded spine member. The mandrel inside the elastomer sleeve (or molded member) 66 is removed, leaving lumen 62 open within the sleeve 66 alongside rib 64. The distal end of the lumen 62 is sealed shut.

When lumen 62 is to be used for irrigation, manifold 60 is attached to the proximal end of the lumen 62 during the process of fabrication. Manifold 60 comprises, for example, a polyimide tube, which is tacked to the proximal end of rib 64, and the elastomer tube is fitted over the distal end of the manifold. Heating the elastomer tube causes it to shrink around manifold 60, as shown in FIG. 6, so that lumen 62 is in fluid communication with manifold 60. Holes are formed by puncturing, drilling or laser drilling through sleeve 66 into lumen 62 to create irrigation outlets 56. One or more electrodes 54 are fitted over and fastened to the outer surface of sleeve 66, for example using a suitable epoxy and/or mechanical fastener, and wires (not shown) are run between the electrodes and insertion tube 25, either along spine 44 or through lumen 62. Irrigation holes 56 can also be provided in electrode 55 itself by forming a hole that extends through the electrode 54 and through the flexible sleeve 66 so that irrigation fluid flows through the manifold 60 through lumen 62 (of sleeve 66) and exits through irrigation hole(s) 56 in electrode 54.

It is noted that the irrigation holes 56 do not have to be at right angles to the rib 64 and can be angled from approximately 45 degrees to 135 degrees with respect to the rib 64 (or longitudinal axis 42). In FIG. 5, one exit hole 56′ (on the electrode 54) is shown as being angled to allow for a desired irrigation flow pattern around the electrode contact surface with body tissue. Other permutations of this feature are within the scope of this invention. For example, the irrigation holes 56 on the sleeve 66 (not on the electrode 54) can all be angled to spray towards the electrode 54 while the irrigation hole 66 on the electrode 54 can be at a right angle to the rib 64. Alternatively, some of the irrigation holes 56 on the sleeve 66 can be angled away from electrode 54 with other irrigation holes 56′ angled towards electrode 54. The electrodes 54 can be configured to have some holes 56′ on the electrode 54 to flow at right angles to the rib 64 (or axis 42) and some holes 56′ at an angle β (referenced by 56′ and axis 42 in FIG. 5) with respect to longitudinal axis 42.

It is noted that holes 56′ may be formed through sleeve 66 (but not through electrode 54) for the purpose of electrically connecting electrode 54 with wire(s) or conductor(s) disposed in lumen 62. The wire(s) disposed in lumen 62 can extend to a proximal handle of the device to allow for signals to flow to and from the electrode(s) 54.

After multiple spines 44 have been fabricated in this manner, the spines are grouped and joined together to form basket assembly 40, which is fixed to the distal end of insertion tube 25 as shown in FIGS. 2A/B.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for producing a medical device, the method comprising:
forming a plurality of spines by:
for each spine, placing a mandrel alongside a rib;
molding a flexible polymer sleeve over the rib and the mandrel; and
after molding the sleeve, removing the mandrel so that the sleeve contains a lumen running parallel to the rib along one side surface of the spine;
fixing one or more electrodes to the sleeve of each of the spines; and
connecting respective proximal ends of the spines together to a distal end of an insertion tube which is configured for insertion into a body cavity of a patient.

2. The method according to claim 1, further comprising conjoining respective distal ends of the spines to form a basket assembly, so that the ribs bow radially outward when the device is deployed in the body cavity, whereby the one or more electrodes contact tissue in the body cavity.

3. The method according to claim 1, wherein ribs are configured to collapse radially inward so that the spines are aligned along an axis of the insertion tube while the device is being inserted into the body cavity.

4. The method according to claim 1, wherein the insertion tube comprises a flexible catheter configured for insertion into a chamber of a heart of the patient, and the electrodes are configured to contact and deliver electrical energy to myocardial tissue within the chamber.

5. The method according to claim 1, wherein the rib comprises a metal slat.

6. The method according to claim 1, wherein the flexible polymer sleeve comprises a thermoplastic elastomer tube.

7. The method according to claim 6, wherein molding the flexible polymer sleeve comprises heating the thermoplastic elastomer tube to a temperature sufficient to cause the thermoplastic elastomer tube to shrink to the shape of the rib and the mandrel.

8. The method according to claim 1, further comprising coupling the lumen of each of the spines to an irrigation manifold running through the insertion tube, and forming irrigation outlets through the flexible polymer sleeve to the lumen in a vicinity of the electrodes, whereby an irrigation fluid passing through the irrigation manifold exits the lumen through the irrigation outlets.

9. The method according to claim 8, wherein the irrigation outlets comprise at least two irrigation outlets in the vicinity of each of the electrodes.

10. The method according to claim 9, wherein a first irrigation outlet of the at least two irrigation outlets in the vicinity of each of the electrodes is configured to direct irrigation fluid toward a respective electrode of the electrodes.

11. The method according to claim 10, wherein a second irrigation outlet of the at least two irrigation outlets in the vicinity of each of the electrodes is configured to direct irrigation fluid away from the respective electrode of the electrodes.

12. The method according to claim 9, wherein the at least two irrigation outlets are formed to produce a desired irrigation flow pattern around each of the electrodes.

13. The method according to claim 1, further comprising passing a wire through the lumen and connecting the wire electrically to at least one of the electrodes.

14. A method for producing a spine for a medical device, the method comprising:
placing a mandrel alongside a resilient rib;
molding a flexible polymer sleeve over the rib and the mandrel; and
after molding the sleeve, removing the mandrel so that the sleeve contains a lumen running parallel to the rib along one side surface of the spine, the spine being configured for attachment to a distal end of an insertion tube such that the lumen aligns with an irrigation manifold of the medical device.

15. The method according to claim 14, wherein the rib comprises a resilient material configured to transition between an expanded configuration and a collapsed configuration of the medical device.

16. The method according to claim 14, wherein the resilient rib comprises a metal slat.

17. The method according to claim 14, wherein the flexible polymer sleeve comprises a thermoplastic elastomer tube.

18. The method according to claim 17, wherein molding the flexible polymer sleeve comprises heating the thermoplastic elastomer tube to a temperature sufficient to cause the thermoplastic elastomer tube to shrink to the shape of the rib and the mandrel.

19. The method according to claim 14, further comprising forming irrigation outlets through the flexible polymer sleeve to the lumen in a vicinity of an electrode, whereby an irrigation fluid passing through the irrigation manifold is configured to exit the lumen through the irrigation outlets.

20. The method according to claim 19, wherein the irrigation outlets comprise at least a first irrigation outlet configured to direct irrigation fluid toward the electrode and a second irrigation outlet configured to direct fluid away from the electrode.
